SEQUENCE LISTING (6 sequences)

SEQ ID NO: 1 - DNA, 21 bp, artificial
  synthetic polynuceotides
tcgtcaagctgctggatgtca

SEQ ID NO: 2 - DNA, 21 bp, Artificial
  synthetic polynucleotides
tgagagcagaggcatccatga

SEQ ID NO: 3 - DNA, 21 bp, artificial
  synthetic polynucleotides
atgtggagcgttggctgtatc

SEQ ID NO: 4 - DNA, 21 bp, artificial
  synthetic polynucleotides
tggaggcaatccaatgagatc

SEQ ID NO: 5 - DNA, 21 bp, artificial
  synthetic polynucleotides
aaggccaaccgtgaaaagatg

SEQ ID NO: 6 - DNA, 21 bp, artificial
  synthetic polynucleotides
aaggaaggctggaaaagagcc

(19) United States
(12) Patent Application Publication
(10) Pub. No.: US 2006/0269568 A1
(43) Pub. Date: Nov. 30, 2006

(54) Reduction of embryonic neural tube defects by pre-or early-administration of germination-activated sporoderm-broken ganoderma lucidum spores to pregnant female

(76) Inventors: Chee-Keung Chung, Kowloon (HK); Yuan-Shan Zeng, Guangzhou (CN)

Correspondence Address: ANDREWS KURTH LLP;Intellectual Property Department, Suite 1100, 1350 I Street, N.W., Washington, DC, 20005, US

(21) Appl. No.: 11/501,881

(22) Filed: Aug. 10, 2006

Related U.S. Application Data
(63) Continuation-in-part of application No. 10/752,685, filed on Jan. 8, 2004.
Application No. 10/752,685 is a continuation-in-part of application No. 10/631,809, filed on Aug. 1, 2003.

(51) Int. Cl. A61K 36/09; A61K 36/06

(52) U.S. Cl. 424/195.15

(57) ABSTRACT

The present invention provides a method for preventing and reducing embryonic neural tube defects (NTDs) caused by cell cycle arrest in the neuroepithelial cells of the embryo. The method comprises administering germination activated sporoderm-broken Ganoderma lucidum spores (“GLSs”) to a female mammal capable of becoming pregnant an effect amount of GLSs. GLSs are preferred to be administered to the female mammal prior to or at an early stage of the pregnancy. The GLSs reduce embryonic NTDs caused by cell cycle arrest in the neuroepithelial cells.

### CROSS REFERENCE TO RELATED APPLICATION

This invention is a continuation-in-part (CIP) of U.S. patent application Ser. No. 10/752,685, filed on Jan. 8, 2004, which in turn is a CIP of U.S. patent application Ser. No. 10/631,809, filed on Aug. 1, 2003, which are herein incorporated by reference.

### FIELD OF THE INVENTION

The present invention relates to a method for preventing and/or reducing embryonic neural tube defects (NTDs). The method comprises administering germination activated sporoderm-broken Ganoderma lucidum spores (“GLSs”) to a female mammal capable of becoming pregnant an effect amount of GLSs. The GLSs are preferred to be orally administered to the female mammal during an early stage of or prior to the pregnancy of the female mammal. GLSs reduce the risk of NTDs caused by cell cycle arrest in the neuroepithelial cells of the embryo.

### BACKGROUND OF THE INVENTION

Neurogenesis is an extremely complex matter, which involves neuroepithelial cell induction, proliferation, migration and differentiation. In the developing vertebrate nervous system, the neural tube is the precursor of the central nervous system, which comprises the brain and the spinal cord. There are two ways in which the neural tube develops: Primary neurulation and secondary neurulation. In primary neurulation, the cells of the neural plate invaginate and pinch off to form the tube. In secondary neurulation, the cells of the neural plate form a cord-like structure that migrates inside the embryo and hollows to form the tube. Each organism uses primary and secondary neurulation to varying degrees. Neurulation in fish proceeds only via the secondary neurulation, while in avian species the caudal regions of the tube develop using secondary neurulation and the anterior regions develop by primary neurulation. In mammals, a similar pattern is observed where secondary neurulation begins around the 35^{th} somite.

The neural tube, for a short time is open both cranially and caudally. These openings, called neuropores, close during the fourth week in humans. Improper closure of the neuropores can result in a neural tube defect (NTD), such as anencephaly or spina bifida.

Anencephaly is a cephalic disorder that results from an NTD. It occurs when the cephalic (head) end of the neural tube fails to close. In humans, it usually occurs between the 23rd and 26th day of pregnancy, resulting in the absence of a major portion of the brain, skull, and scalp. Infants with this disorder are born without a forebrain—the largest part of the brain consisting mainly of the cerebrum—which is responsible for thinking and coordination. The remaining brain tissue is often exposed—not covered by bone or skin.

Infants born with anencephaly are usually blind, deaf, unconscious, and unable to feel pain. Although some individuals with anencephaly may be born with a rudimentary brainstem, which controls autonomic and regulatory function, the lack of a functioning cerebrum permanently rules out the possibility of ever gaining consciousness. Reflex actions such as respiration (breathing) and responses to sound or touch may occur. The disorder is one of the most common disorders of the fetal central nervous system.

Most of the fetuses who are affected by anencephaly die in the womb of the mother, during childbirth or in the next few hours after exited the mother's body. In some cases, however, it has been known that certain infants have lived up to a week and a half, and the current record lifespan is twelve years.

In the United States, approximately 1,000 to 2,000 babies are born with anencephaly each year. Female babies are more likely to be affected by the disorder. About 95% of women who learn that they will have an anencephalic baby choose to have an abortion. Of the remaining 5%, about 55% are stillborn. The rest usually live only a few hours or days.

Spina bifida is a Latin term which means “split spine.” Spina bifida is caused by the failure of the neural tube to close during embryonic development. In humans, normally the closure of the neural tube occurs around the thirtieth day after fertilization. However, if something interferes and the tube fails to close properly, a neural tube defect will occur. An incomplete closure of one or more vertebral arches of the spine, resulting in malformations of the spinal cord. The spinal membranes and spinal cord may protrude through the absence of vertebral arches (called clefts).

Spina bifida has varying prevalence in different human populations. This and extensive evidence from mouse strains with spina bifida suggests a genetic basis. As with other human diseases such as cancer, hypertension and atherosclerosis (coronary artery disease), spina bifida likely results from the interaction of multiple genes and environmental factors. Despite much research it's still unknown what causes the majority of cases. It is important to note that spina bifida occurs by the 4^{th} week of pregnancy in humans before many women will be aware of a pregnancy.

The most common locations of the malformations in humans are in the lumbar and sacral areas. The lumbar nerves control the muscles in the hip, leg, knee and foot, and help to keep the body erect. The sacral nerves control some of the muscles in the feet, bowel and bladder and the ability to have an erection. Some degree of impairment can be expected in these areas.

In humans, spina bifida results in varying degrees of paralysis, absence of skin sensation, incontinence, and spine and limb problems depending on the severity and location of the lesion damage on the spine. In very rare cases, cognitive problems also result.

Most babies born with the condition will need surgeries to correct spinal, foot or leg problems, shunt surgery to drain fluid from the brain, application of techniques to control bladder and bowel function (such as self-catherization or diapers), and braces or other equipment to assist in walking.

In Canada, spina bifida occurs in about one in every 1000 births. In Western Australia, up until 1996, around 2 children in every 1000 were born with a neural tube defect. Since 1996, the figure has dropped to 1.3 children per 1000 births. In the United States, spina bifida occurs in about 7 out of 10,000 live births. More children in the U.S. have spina bifida than have muscular dystrophy, multiple sclerosis, and cystic fibrosis combined.

Ganoderma belongs to the Polyporaceae group of the Fungi family. Ganoderma is widely used in traditional Chinese medicine as an auxiliary treatment for a variety of medical conditions, such as hepatis, AIDS, cancer, and autoimmune diseases. The Ganoderma spores are the essence of Ganoderma and contain active ingredients such as polysaccharides, sterols, oleic acid, linoleic acid, triterpenes, ceramides and certain organic ions, all of which possessing strong bioactivity. In the parent U.S. patent application Ser. No. 10/631,809 and 10/752,685, which are herein incorporated by reference, the Ganoderma spores have shown activities in treatment of neurological disorders, including spinal cord injury, and can promote neural stem cells proliferation and/or differentiation in injured spinal cord. To date, however, there have been no reports on the effects of Ganoderma spores on the occurrence of NTD during pregnancy.

### SUMMARY OF THE INVENTION

The present invention provides a method for reducing neural tube defects (NTDs) in mammalian embryos and/or fetuses. The method comprises administering to a female mammal capable of becoming pregnant an effective amount of germination activated sporoderm-broken Ganoderma lucidum spores (GLSs). It is preferred to orally administer GLSs to the female mammal prior to or at the early stage of the pregnancy. The NTD is preferred to be caused by cell cycle arrest, preferably at the G₀/G₁ phase of the cell cycle, of the neuroepithelial cells in the embryo. For example, a cell cycle arrest is caused by inhibition of cyclin-dependent kinase (Cdk), such as Cdk4.

The most common forms of NTD include anencephaly and spina bifida.

When the female mammal is a human, GLSs are preferred to be administered prior to the pregnancy or in any event, no later than 4 weeks of her pregnancy.

When the female mammal is a rodent, such as a mouse, GLSs are preferred to be administered prior to the pregnancy or in any event, no later than 10.5 days of her pregnancy.

One way to induce cell cycle arrest in the neuroepithelial cells of the embryo is by treating the female mammalian retinoic acid, such as all trans retinoic acid (ATRA).

GLSs are prepared by (1) soaking Ganoderma spores in a solution which is selected from the group consisting of water, saline, and a nutritional solution to cause the spores to germinate; (2) placing the germination-treated Ganoderma spores in a culture box at a relative humidity of 65-98% and temperature of 18-48° C. to cause the germinated Ganoderma spores to activate; and (3) breaking sporoderm of the germination activated Ganoderma spores to produce the GLSs.

GLSs are preferred to be administered orally. The preferred daily dose of GLSs is between 0.01 and 20 g/ kg body weight, and more favorably, between 0.1 and 20 g/kg body weight. GLSs increase Cdk4 expression in the neuroepithelial cells of the embryo. GLSs also promote proliferation and differentiation of the neuroepithelial cells in the embryo.

GLSs increases the expression of Cdk4 in the neuroepithelial cells of the embryos and promote the proliferation and differentiation of the neuroepithelial cells.

### BRIEF DESCRIPTION OF DRAWINGS

FIG. 1A is a fluorescence microscopic picture of a E10.5 day mouse embryo from the normal control group. The horizontal red line across the head portion of the embryo represents the cross-sectional area shown in FIGS. 1B-1E.

FIG. 1B is a fluorescence microscopic picture showing the state of the embryo's coronal plane (fluorescence double-labeled nestin (red) and Hoechst 33342 (blue)).

FIGS. 1C-1E are fluorescence microscopic pictures of nestin immunofluorescence staining of the same mouse embryo section. The scale bar (the white line at the bottom right corner of the picture) equals 1 mm in FIGS. 1B and 1C, 0.25 mm in FIG. 1D, and 0.05 mm in FIG. 1E.

FIG. 2A is a fluorescence microscopic picture of a E10.5 day mouse embryo from the experimental control group. The horizontal red line across the head portion of the embryo represents the cross-sectional area shown in FIGS. 2B-2E.

FIG. 2B is a fluorescence microscopic picture showing the state of the embryo's coronal plane (fluorescence double-labeled nestin (red) and Hoechst 33342 (blue)).

FIGS. 2C-2E are pictures of nestin immunofluorescence staining of the same mouse embryo section. The scale bar (the white line at the bottom right corner of the picture) equals 1 mm in FIGS. 2B and 1C, 0.25 mm in FIG. 2D, and 0.05 mm in FIG. 2E.

FIG. 3A is a fluorescence microscopic picture of a E10.5 day mouse embryo from the GLSs group. The horizontal red line across the head portion of the embryo represents the cross-sectional area shown in FIGS. 3B-3E.

FIG. 3B is a fluorescence microscopic picture showing the state of the embryo's coronal plane (fluorescence double-labeled nestin (red) and Hoechst 33342 (blue)).

FIGS. 3C-3E are pictures of nestin immunofluorescence staining of the same mouse embryo section. The scale bar (the white line at the bottom right corner of the picture) equals 1 mm in FIGS. 3B and 3C, 0.25 mm in FIG. 3D, and 0.05 mm in FIG. 3E.

FIG. 4 is a diagram showing flow cytometry analysis of nestin expression in embryonic neural tube epithelial cells. Red: Experimental control group; Blue: Normal control group; Green: GLSs group. The vertical axis: the number of cells; the horizontal axis: intensity of the fluorescence.

FIG. 5 is a picture showing the Cyclin-dependent kinase 2 (Cdk2) RT-PCR products after electrophoresis in agarose gel. M-marker; 1-blank control group; 2-normal control group; 3-Experimental control group; 4-GLSs group.

FIG. 6 is a picture showing the Cyclin-dependent kinase 4 (Cdk4) RT-PCR products after electrophoresis in agarose gel. M-marker; 1-blank control group; 2-normal control group; 3-Experimental control group; 4-GLSs group.

FIG. 7 is a composite of fluorescence microscopic pictures showing Cdk4 expression in E10.5 day mouse embryos. Panel A1-A3, embryo sections from the blank group, amplification 25×, 50× and 100×, respectively. Panel B1-B3, embryo sections from the normal control group, amplification 25×, 50× and 100×, respectively. Panel C1-C3, embryo sections from the experimental; control group, amplification 25×, 50× and 100×, respectively. Panel D1-D3, embryo sections from the GLSs group, amplification 25×, 50× and 100×, respectively.

### DETAILED DESCRIPTION OF THE INVENTION

All organisms consist of cells that multiply through cell division. Before a cell can divide, it has to grow in size, duplicate its chromosomes and separate the chromosomes for exact distribution between the two daughter cells. These different processes are coordinated in the cell cycle. In a mammalian cell, the cell cycle consists of several phases. In the first phase (G₁), the cell grows and becomes larger. When it has reached a certain size it enters the next phase (S), in which DNA-synthesis takes place. The cell duplicates its hereditary material (DNA-replication) and a copy of each chromosome is formed. During the next phase (G₂), the cell checks that DNA-replication is completed and prepares for cell division. The chromosomes are separated and the cell divides into two daughter cells. Through this mechanism, the daughter cells receive identical chromosome as in the parent cells. After division, the cells are back in G₁ and the cell cycle is completed. However, cells in the first cell cycle phase (G₁) do not always continue through the cycle. Instead, they can exit from the cell cycle and enter into a resting stage (G₀), also known as G₀/G₁ arrest. The biochemical events that regulate the proliferation/differentiation (P/D) transition at G₁ exit are unclear at this time.

A group of the key regulators of the cell cycle, cylin-dependent kinases (Cdks), was discovered by Leland H. Hartwell, R. Timothy Hunt, and Paul M. Nurse, the 2001 Nobel Prize Laureates in Physiology or Medicine. Different Cdks control the major steps between different phases of the cell cycle through phosphorylation of cell proteins like histones, cytoskeletal proteins, transcription factors, tumor suppresser genes and others. Hence all external signals related to cell growth and division must eventually regulate the activity of one of the Cdks. This mechanism has been so well conserved during evolution that for example human regulatory proteins can successfully be incorporated and regulate the cell cycle of yeast cells. Cdks phosphorylate proteins are Serine/Threonine kinases. Nigg, E E, Curr. Opin. Cell Biol., 5: 187-193(1993). A Cdk is activated by association with a cyclin forming a cyclin-Cdk complex.

Recent reports have shown that the group of cyclin/Cdk complexes is required for cell cycle progression during G₁/S transition. Cyclin D/Cdk4/6 complexes function early in G₁ and act as the primary sensors of positive and negative environmental signals. Sherr C J & Roberts, J M, Genes Dev., 13: 1501-1512 (1999). Extracellular factors impinge primarily on Cdks to arrest cells in the G₁ phase of the cell cycle. Wainwright L J et al., Proc. Natl. Acad. Sci., 98(16):9396-9400 (2001). Mechanisms for the inhibition of cyclin D-Cdk4/6 include regulation of cyclin production and binding to the Cdks, subunit phosphorylation, and inhibition by Cdk inhibitors. Sherr C J, Cell 79:551-555 (1994). In mammalian cells, the cooperation between two classes of Cdk inhibitors, the Cdk inhibitor protein/kinase inhibitor protein (Cip/Kip) and the Cdk4 inhibitor (Ink4) family of inhibitors, is frequently responsible for withdrawal from the cell cycle. Sherr C J & Roberts, J M, Genes Dev., (1999), supra.

Retinoic acid is an important regulator of normal cellular proliferation and differentiation and suppressor of tumor growth by cell cycle arrest and apoptosis. Recent report demonstrates that retinoic acid induces cell cycle arrest and differentiation in human neuroblastoma (NB) cells. Wainwright U et al., Proc. Natl. Acad. Sci., (2001), supra.

Retinoic acid may act as a regulator of differentiation at various stages of vertebrate embryogenesis. In particular, the results of exogeneous retinoic acid treatment have implicated retinoic acid in antero-posterior patterning both along the body axis and in developing limb bud. A variety of abnormalities, including neural tube defects, craniofacial abnormalities, and limb defects, have been reported after treatment of pregnant mammals with retinoic acid. Spirov, A V et al., Nucleic Acids Research, 28: 337-340 (2000). According to another recent report, retinoic acid is a necessary component for normal differentiation during the secondary neurulation. Excess RA, however, is teratogenic and causes NTDs. Griffith M. et al., Teratology, 62(2): 123-133 (2000). Although the way in which retinoic acid modulates secondary neurulation is unclear, research has shown that excess retinoic acid inhibits the expression of Cdk2 and Cdk4 (both are key factors that allow cells to progress into the S/G2(2)+M phase of the cell cycle normally), thus causing embryonic neuroepithelial cells to be arrested in the G₀/G₁ phase of the cell cycle, which in turn further reduces the rate of cell proliferation and/or differentiation, and reduces the number of neural stem cells, leading to abnormal development of the neural tube, which causes NTDs. Wang L. et al., Develop. Growth Differ., 47(3):119-130 (2005); Wang J. et al., J. Biol. Chem., 277(45): 43369-43376 (2002); Sarkar S A and Sharma R P, Cell Biol. Toxicol., 18(4): 243-257 (2002).

In describing preferred embodiments of the present invention, specific terminology is employed for the sake of clarity. However, the invention is not intended to be limited to the specific terminology so selected. It is to be understood that each specific element includes all technical equivalents which operate in a similar manner to accomplish a similar purpose.

One aspect of the present invention relates to a method for preventing or reducing NTDs in mammalian embryos/fetuses. The method comprises administering to a female mammal capable of becoming pregnant an effective amount of GLSs.

The female mammal can be any mammal, including but not limited to, human, pets such as dogs and cats, farm animals such as sheep, goat, cow, and pig, and laboratory animals such as mouse, rat, guinea pig, rabbit, monkey and baboon. The female mammal is at an offspring- or a child-bearing age.

GLSs are brown powders that are slightly soluble in water. Briefly, bioactive GLSs can be produced in a process containing the following steps:

I. Induction of germination: Mature and perfect spores of Ganoderma lucidum are carefully selected to undergo a soaking process to induce germination. Spores are kept in clear or distilled water, biological saline solution, or other nutritional solutions that could enable the spores of Ganoderma lucidum to germinate rapidly. Examples of nutritional solutions include coconut juice or a 1-5% malt extract solution, 0.5-25% extracts of Ganodenna lucidum sporocarps or Ganoderna lucidum capillitia, 0.1-5% of culture solution containing biotin, 0.1-3% of culture solution containing potassium phosphate (monobasic) and magnesium sulfate. The choice of solution would depend on the soaking time required, the amount of spores to be processed and other such factors as availability of materials. One or more of the above germination solutions could be used, with the amount added being 0.1-5 times the weight of the spores of Ganoderma lucidum. The soaking time can be determined according to the temperature of the water, and usually the soaking was carried out for 30 min to 8 hours with the temperature of the water at 20-43° C. Preferably, the soaking time is 2-4 hours, and the temperature of water is 25-35° C.

II. Activation culture: The spores of Ganoderma lucidum are removed from the soaking solution and excess solution is eliminated by allowing it to drip. The spores are then placed in a well-ventilated culturing box at a constant temperature and humidity so that spore culture activation could be carried out. The relative humidity of the culture was generally set at 65-98%, the culture temperature set at 18-48° C. and the activation time lasted from 30 min to 24 hours. Preferably humidity is 85-97% and temperature is 25-35° C. During activation, the cell walls of the spores of Ganoderma lucidum are clearly softened such that it was easier to penetrate the cell walls of the spores. The activation of spores of Ganoderma lucidum typically reaches a rate of more than 95%.

III. Treatment of the epispores: After the germination/activation process, the spores are treated by enzymolysis. This process is carried out at a low temperature and under conditions such that enzyme activity is maintained, using chitinase, cellulase, or other enzymes, which are commonly used in the industry. The process is complete when the epispores lost their resilience and became brittle. Alternatively, physical treatments are carried out to penetrate the cell walls, for example, micronization, roll pressing, grinding, super high pressure microstream treatment, and other mechanical methods commonly used in the industry could be carried out, with a penetration rate of over 99%.

IV. Drying or extraction: Drying is carried out at low temperature using standard methods including freeze-drying or vacuum-drying etc., which are commonly used in the industry. The obtained product has a moisture content less than 4%. After drying, the bioactive substances are extracted by water or alcohol, or by thin film condensation. The extracted bioactive substances can be further purified by dialysis to ensure no contamination in the final products. The final product can be made into purified powders, extract pastes, solutions for injection, or for oral consumption.

A more detailed description for production of GLSs can be found in U.S. Pat. No. 6,316,002, which is hereby incorporated by reference. GLSs are also commercially available from Kindway/Holistol International Limited in Hong Kong.

The effective amount of GLSs is a dosage which is useful for preventing or reducing NTDs in a target embryo/fetus. Toxicity and therapeutic efficacy of GLSs can be determined by standard pharmaceutical procedures in cell culture or experimental animal models, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The data obtained from cell culture assays and animal studies can be used in formulating a range of dosages for use in humans.

Generally, appropriate dosages for administering GLSs may range, for example, from about 0.01 g/kg body weight/day to about 20 g/kg body weight/day. In one embodiment, the effective amount of GLSs is between 0.1 and 20 g/kg body weight/day. In another embodiment, the effective amount of GLSs is between 1 and 10 g/kg body weight/day. In yet another embodiment, the effective amount of GLSs is between 8 g/kg body weight/day.

GLSs are preferred to be administered orally. The administration can be in one dose, or at intervals such as three times daily, twice daily, once daily, once every other day, or once weekly. Dosage schedules for administration of GLSs can be adjusted based on the individual conditions and needs of the target. Continuous infusions may also be used after the bolus dose. The effects of any particular dosage can be monitored by suitable bioassays.

GLSs can also be formulated into a pharmaceutical composition with a pharmaceutically acceptable carrier. As used herein the language “pharmaceutically acceptable carrier” is intended to include any and all solvents, solubilizers, fillers, stabilizers, binders, absorbents, bases, buffering agents, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary agents can also be incorporated into the compositions.

The pharmaceutical composition of the present invention is formulated to be compatible with its intended route of administration, e.g., oral or parenteral administration. Oral compositions generally include an inert diluent or an edible carrier. They can be enclosed in gelatin capsules or compressed into tablets. For the purpose of oral therapeutic administration, the active compound can be incorporated with a solid carrier and used in the form of tablets, troches, or capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

Alternatively, GLSs can be formulated for injection. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, such as sodium chloride, sugars, polyalcohols (e.g., manitol, sorbitol, etc.) in the composition. Prolonged absorption of the injectable compositions can be brought about by including in the composition an agent which delays absorption, for example, aluminum monostearate and gelatin.

Sterile injectable solutions can be prepared by incorporating the GLSs in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate oral or parenteral compositions in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

The following experimental design is illustrative, but not limiting the scope of the present invention. Reasonable variations, such as those occur to reasonable artisan, can be made herein without departing from the scope of the present invention. Also, in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### Experimental Design

I. Materials and Methods

Pure Kunming line adult mice weighing 30-40 g were purchased from the Guangdong Province Experimental Animal Center (Guangdong Documentation Number 2004A019). At 6 p.m. the animals were paired at a 1:1 male/female ratio and placed in cages together. At 8 am the next day, the female mice were checked for vaginal plugs. Mice having vaginal plugs were defined as pregnant at E0 day pregnancy, and at E0.5 day pregnancy by 4 pm the same day. At E0 day, the pregnant mice were randomly placed into four groups: the blank control group, the normal control group, the experimental control group and the Ganoderma spores group.

All-trans retinoic acid, nestin, and Hoechst 33342 were purchased from Sigma, (St Louis, MS, USA). Carboxymethylcellulose solution was purchased from Guangzhou City Medicine Company (Guangzhou, China) in import packaging, license number 96050. RNAlater was purchased from Tianwei Modern Biological Reagent Company (Beijing, China), Ganoderma spores were provided by Kindway/Holistol International Ltd. in Hong Kong (license number GANSPSP04040104) as Ganoderma spores with fully broken walls (2036).

Ganoderma spores treatment. At E0 day the pregnant mice in the GLSs group were administered GLSs solution (0.5% methylcellulose preparation) by gastric feeding at a dosage of 8 g/kg body weight/day (0.5ml/administration, twice daily) until sacrifice. Pregnant mice in the normal control group and the experimental control group received an equivalent dose solution by gavage. At E7.75 days, all-trans retinoic acid (ATRA) was administered at a dose of 50 mg/kg body weight once via gavage to the mice in the experimental control group and the GLSs group. The administration of ATRA is according to Moase and Trasler, Teratology, 36 335 -343 (1987). The mice were sacrificed at E10.5 days. The pregnant mice in the blank control group did not receive any treatment.

Sample preparation. At sacrifice, embryos were collected and examined using dissection microscopy. The incidence of NTDs in the four groups of mouse embryos was determined. The mouse embryos were marked at the cerebral vesicle crown and the tail base, and the distance between the two points measured to serve as the crown-to-rump distance. For each group of pregnant mice, a set quantity of mouse embryos was fixed in a 4% paraformaldehyde solution. Cross sections of the embryos (section thickness was 20 μm) were prepared with a freezing microtome. Neural tubes were isolated from each of the four groups of mouse embryos and divided into aliquots. For each group, an aliquot of neural tubes was placed into an RNAlater containing a RNAse inhibitor and stored at −30° C.; another aliquot of neural tubes was sheared into pieces, triturated using pipettes in PBS, and filtered through a 200-mesh filter to form a single-cell suspension. The cells were fixed in 75% alcohol (4° C.).

Nestin immunofluorescence histochemical staining. The embryos in the cross sections were incubated in 0.3% Triton X-100 for 15 min at room temperature, blocked by serum for 20 min, and incubated with murine anti-nestin antibody (1:1000) at 4° C. overnight. The next day, the sections were subjected to SABC-cy3 development for 20 min and counter stained with Hoechst 33342 for 1 h. After washing with 0.01M PBS, the slide was sealed and placed under a fluorescence microscope for observation. The negative control slides were incubated with PBS instead of the murine anti-nestin antibody.

Nestin immunofluorescent staining of suspended cells. Cell suspension obtained from the embryonic neural tubes was centrifuged to remove the supernatant. The pellet was resuspended in an IC fixation buffer (eBioscience, USA) and incubated for 15 min. The fixed cells were pelleted after centrifugation, resuspended in 300 μl of a permeabilization buffer (eBioscience, USA), and incubated for 10 min. The cells were rinsed twice with PBS, incubated with sheep serum (Santa Cruz, USA, 1:100) at room temperature for 15 min, then incubated with murine anti-nestin antibody (1:1000) at 37° C. for 30 min. The cells were then rinsed with PBS, incubated with sheep anti-mouse IgG-FITC (Santa Cruz, USA 1:50) at 37° C. for 30 min, rinsed with PBS again, and resuspended at a concentration of 5×10⁶ cell/ml. Cells from the negative control group was not incubated with the anti-nestin antibody and was incubated directly with sheep anti-mouse IgG-FITC.

Propyl isoferulate (PI) defined DNA fluorescent staining: Single cell suspension of neural tubes was centrifuged to remove the supernatant. The cells were resuspended in RNase (0.1 mg/l), incubated at 37° C. for 30 min, rinsed with saline, and then incubated with PI stain (0.05 mg/l, 0.03% Triton X-100) at 4° C. for 30 min in an environment protected from light. [0012] RT-PCR. Total RNA from fresh mouse embryonic neural tubes was extracted using Trizol (Life Technologies, USA). 2 μg of total RNA was used to synthesize cDNA with AMV reverse transcriptase (Promege USA). The Cdk2 upstream and downstream primers were 5′-TCGTCAAGCTGCTGGATGTCA-3′ (SEQ ID NO:1) and 5′-TGAGAGCAGAGGCATCCATGA-3′ (SEQ ID NO:2), respectively. The amplification product was 102 bp. The Cdk4 upstream and downstream primers were 5′-ATGTGGAGCGTTGGCTGTATC-3′ (SEQ ID NO:3) and 5′-TGGAGGCAATCCAATGAGATC-3′ (SEQ ID NO:4), respectively. The amplification product was 114 bp. The β-actin's upstream and downstream primers were 5′-AAGGCCAACCGTGAAAAGATG-3′ (SEQ ID NO:5) and 5′-AAGGAAGGCTGGAAAAGAGCC-3′ (SEQ ID NO:6). The amplification product was 461 bp. PCR amplification conditions for all primers were: denaturation at 93° C. for 5 min; amplification at 93° C. for 30 sec; 57° C. for 30 sec; and 72° C. for 30 sec for 35 cycles; and extension at 72° C. for 10 min. The PCR products were detected by agarose gel electrophoresis followed with a bromide stain. A UVP gel imaging system was used to capture the images.

Flow cytometry and statistical processing. A Beckman-Coulter's Elite flow cytometer and corresponding software were used to obtain the cell counts. Each specimen was adjusted using the same voltage as the negative control, limited to 1% of its positive expression rate. then Fluorescence of embryonic neural tube cells from the four groups were measured. Nestin-positive cells were counted and corresponding graphs were drawn. Each group of specimens had five tubes and 10,000 cells were measured in each specimen. The results were shown as the percentage of positive cells. DNA cycle analysis was performed with lasers at wavelengths of 488 nm. Phoenix Company's Multicycle software was used to process collected cells and calculate percentage of cells at various stages of cell cycle. The cell cycle data was then analyzed using χ² test and single factor variance analysis. A SPSS10.0 statistical software was used to process the data.

II. Result

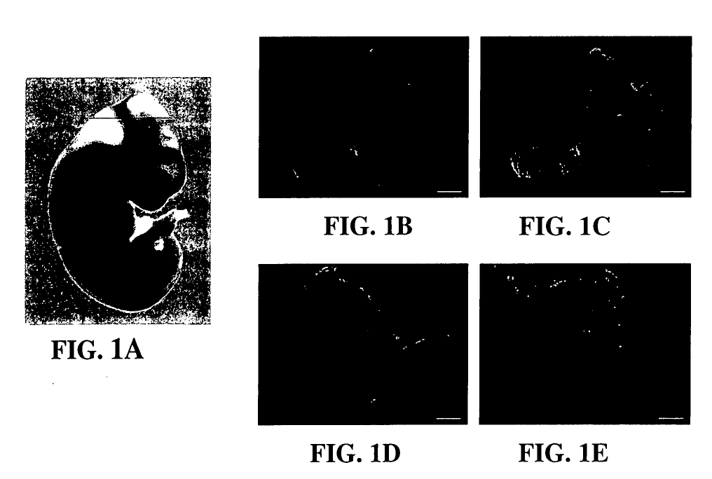

Embryo observations, NTD incidence and crown-to-rump distance. The blank and normal control groups had mostly live embryos at E10.5 days, with an occasional dead embryos. The cerebral vesicles in the heads of the live embryos were completely closed (FIG. 1), and the embryos had a fuller appearance. The optic vesicles, otic vesicles, branchial arches, anterior and posterior limb buds, and about 25-30 protovertebral segments were visible in these embryos. The surface of the spines were smooth and free of cracking, and the tails were curved and long.

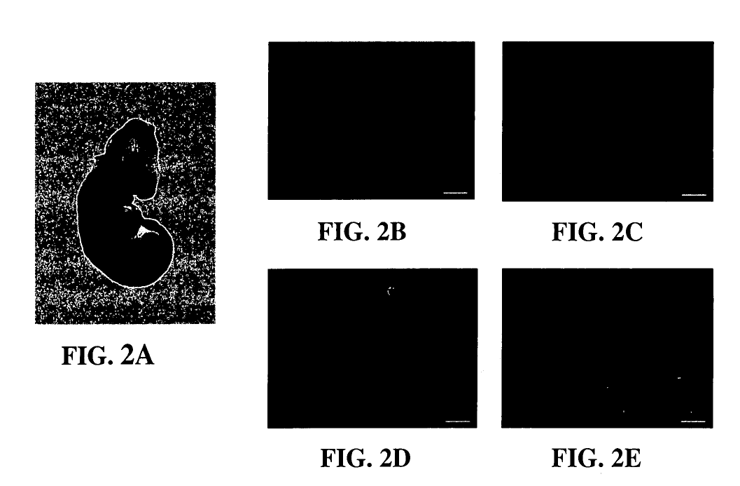

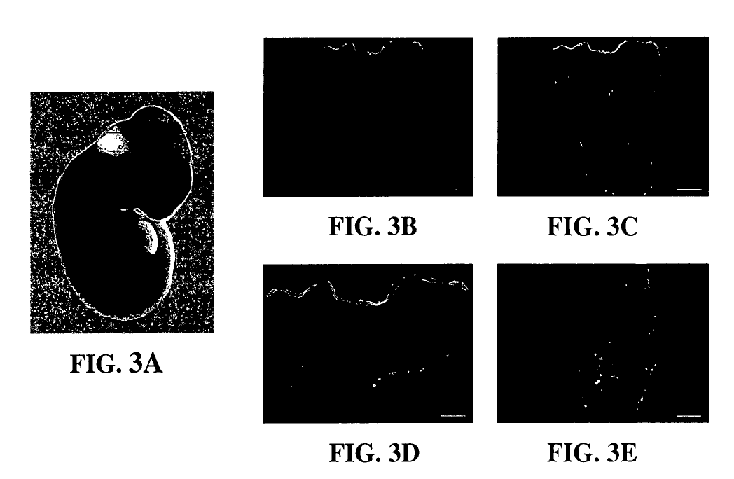

Embryos from the experimental control group had a clearly greater number of dead embryos, and various NTDs were seen (for example, anencephaly and lack of craniospinal fusion was shown in FIG. 2; spina bifida was also observed (not shown in the figure)). The facial shapes were abnormal and the tails were short or non-existent. Embryos from the GLSs group were basically normal in appearance, except for the presence of an extremely small number of NTDs (FIG. 3).

With respect to incidence of NTDs (Table 1) among the four groups, while the difference between the blank and normal control groups was statistically insignificant, the differences among the remaining groups were more significant (p<0.01). In terms of crown-to-rump distance (Table 2), differences among the groups were statistically significant (p<0.05), except for the difference between the blank and normal control groups.

Nestin expression in embryonic neural tube neuroepithelial cells. Nestin refers to a member of the family of intermediate filaments and comes from the fact that this protein is expressed mainly in neuroepithelial stem cells. Nestin is not expressed in mature elements and terminal cell differentiation is associated with loss of immunoreactivity to this protein. Therefore, immunohistochemical assessment of nestin expression is used to differentiate between mature and immature elements

In the blank and normal control groups, nestin was expressed in neuroepithelial cells near the border membrane in the murine embryonic neural tubes at E10.5 days. The nestin positive cells were distributed within the entire neural tube (FIGS. 1A-1D).

The neuroepithelial cells in the experimental control group had lower nestin expression than that of the blank and normal control groups. In addition, the distribution of nestin positive neuroepithelial cell was more restricted. Only a weak expression was detected in the neuroepithelial cells in the defective area (FIGS. 2A-2D).

Nestin expression in neural tube neuroepithelial cells of the GLSs group does not differ significantly from those in the blank control group. The nestin positive neuroepithelial cells were distributed throughout the entire neural tube (FIGS. 3A-3D).

TABLE 1
Comparison of NTDs Rates of E10.5 Day Embryos Among Four
Experimental Groups

| | | | Dead | Abnormal | |
|---|---|---|---|---|---|
| | | Total | embryos | embryos | |
| Group | n | embryos | % | | NTDs rate |
| Blank control | 5 | 53 | 2 | 0 | 3.77%*^{Δ} |
| Normal control | 5 | 62 | 5 | 0 | 8.62%*^{Δ} |
| Experimental | 5 | 68 | 20 | 34 | 79.41%* |
| control | | | | | |
| GLSs | 5 | 60 | 7 | 6 | 21.67%^{Δ} |

X² test: *P < 0.05, vs GLSs Group, ^{Δ}P < 0.05, vs Experimental Control Group

TABLE 2
Comparison of Crown-Rump Distance of E10.5 Day
Embryos Among Four Experimental Groups

| | Blank | Normal | Experimental | |
|---|---|---|---|---|
| | control | control | control | GLSs |
| Group | (\overline{x} ± s, mm) | | | |
| n | 5 | 5 | 5 | 5 |
| Crown-rump | 5.62 ± 0.44^{Δ} | 5.96 ± 0.55^{Δ} | 3.62 ± 1.27* | 5.84 ± 0.92^{Δ} |
| distance | | | | |

one way ANOVA: *P < 0.05, vs GLSs Group, ^{Δ}P < 0.05, vs Experimental Control Group

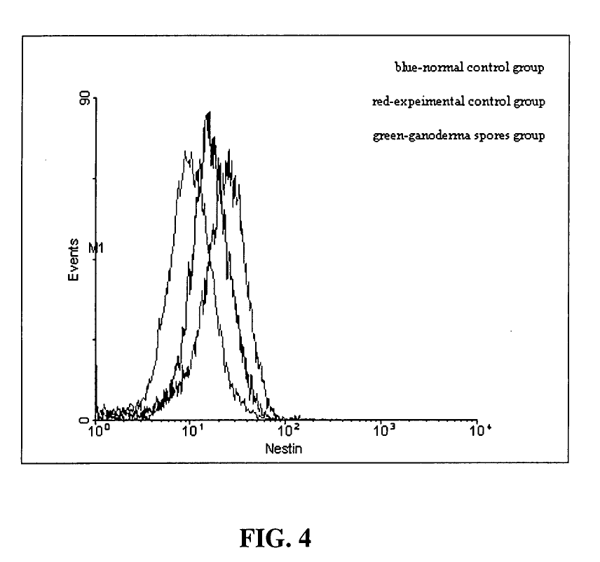

Percentage of nestin-positive embryonic neural tube neuroepithelial cells and percentage of neural tube cells at various phases of cell cycle. Nestin-positive cells were detectable in the neural tubes of E10.5 day mouse embryos from all four groups. Compared to those in the normal control group, the nestin-positive cell counts from the experimental control group were clearly lower. The nestin-positive cell counts from the GLSs group, however, were close to and even higher than those from the normal control group (See FIG. 4).

Among the four groups, the experimental control group had the lowest nestin-positive percentage of embryonic neural tube epithelial cells. There was no significant difference (p>0.05) in nestin positive cell percentages among the other three groups (see Table 3). The percentage of G₀/G₁ phase embryonic neural tube epithelial cells in the GLSs group was clearly lower than those of the experimental control group (p<0.05, Table 4), the blank group, and the normal control group. The percentage of S phase embryonic neural tube epithelial cells in the GLSs group was clearly higher than those in the other control groups (p<0.05).

TABLE 3
Percentage of Nestin Positive E10.5d Embryonic Neural Tube
Epithelia Cells Among Four Experimental Groups

| | Blank control | Normal control | Experimental control | GLSs |
|---|---|---|---|---|
| Group | (\overline{x} ± s, %) | | | |
| n | 5 | 5 | 5 | 5 |
| Positive ratio | 74.73 ± 10.49* | 77.83 ± 18.88* | 32.44 ± 4.64^{Δ} | 77.65 ± 5.81* |

one way ANOVA: *P < 0.05, vs Experimental control group ^{Δ}P < 0.05, vs GLSs group

TABLE 4
Percentage of E10.5d Embryonic Neural Tube Epithelia Cells Stagnated
In Different Phase of Cell Cycle Among Four Experimental Groups

| | | G₀/G₁ | G₂/M | S |
|---|---|---|---|---|
| Group | n | (\overline{x} ± s, %) | | |
| Blank control | 5 | 65.62 ± 10.99*^{Δ} | 6.60 ± 4.40 | 27.76 ± 12.20*^{Δ} |
| Normal control | 5 | 65.94 ± 1.90*^{Δ} | 10.02 ± 3.90* | 24.02 ± 5.26*^{Δ} |
| Experimental control | 5 | 82.80 ± 7.39^{Δ} | 4.00 ± 2.13 | 13.20 ± 5.35^{Δ} |
| Ganoderma spore | 5 | 53.74 ± 3.15* | 7.12 ± 3.73 | 39.18 ± 4.05* |

one way ANOVA: *P < 0.05, vs Experimental Control group ^{Δ}P < 0.05, vs Ganoderma spore group

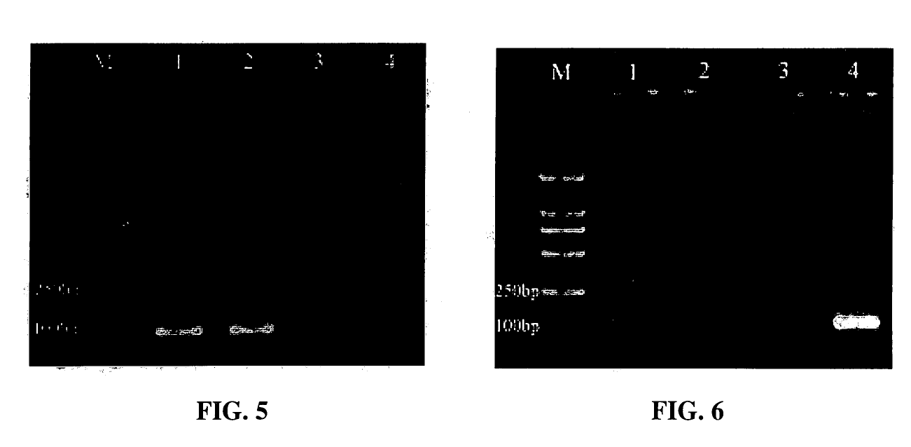

Transcription of Cdk2 mRNA and Cdk4 mRNA. Agarose gel electrophoresis was performed on the Cdk2 RT-PCR products. A specific band of about 102 bp was observed in the blank and normal control groups and there was no clear difference in the fluorescence intensity between bands from the two groups. Such bands was not observed in the GLSs group and the experimental control group (FIG. 5). Electrophoresis performed on the Cdk4 RT-PCR products showed a high intensity band of about 114 bp in the Ganoderma spores group. This band was also observed in other three groups, with a lower intensity in the blank and normal control groups, and the lowest intensity in the experimental control group (FIG. 6).

Expression of Cdk4 in E10.5 day embryos. E10.5 day embryos from the four experimental groups were examined for Cdk4 expression by immunohistochemistry. The embryo tissues were fixed, embedded and frozen in an embedding medium, and sectioned at 20 μm thickness. Cdk4 immunofluorescence histochemical staining was performed on coronal plane sections and examined under a fluorescence microscope.

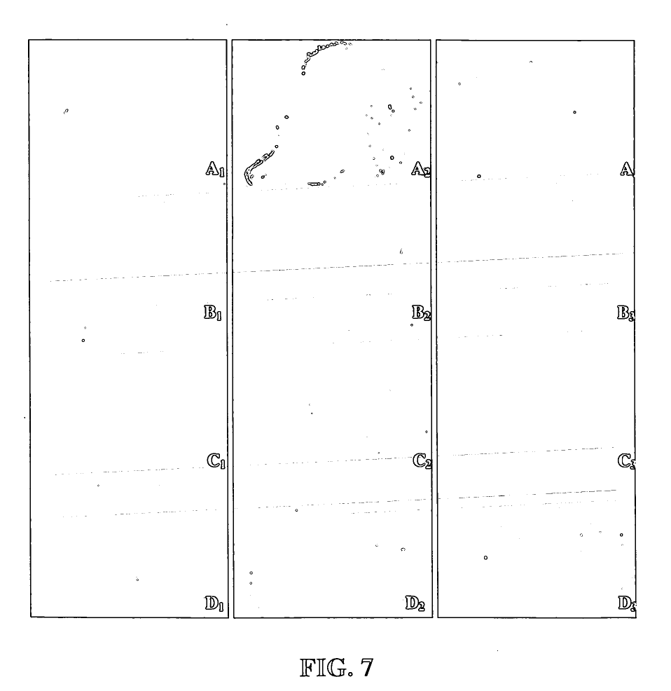

As shown in FIG. 7, tissue cells formed in the ectoderm and the mesoderm of the coronal plane section of mouse embryos all show Cdk4 positive staining. However, neuroepithelial cells in the neural tube show stronger Cdk4 staining. Because embryonic mice develop in stages and there are other causative factors, the objective of this experiment is chiefly to observe Cdk4 expression in the neural tube. In all four groups of mouse embryos, Cdk4 positive cells are evenly distributed within the entire neural tube, and the difference lies only in the intensity of the Cdk4 staining.

Embryos from the blank and normal control groups showed strong Cdk4 staining in the neural tubes (FIG. 7, panel A1-A3 and panel B1-B3). Embryos from the experimental control group (treated with a single dose of retinoic acid), however, showed much weak staining of Cdk4 in the embryonic neural tubes (FIG. 7, C1-C3), indicating that retinoic acid treatment reduces Cdk4 expression in the neural tube epithelial cells of embryonic mice. In the group that received a preadministration of GLSs, Cdk4 staining (FIGS. D1-3) of the neural tube epithelial cells was as strong as, or even higher than that of the blank and normal control groups, indicating that preadministration of GLSs restore Cdk4 expression in the neural tube epithelial cells of embryonic mice treated with retinoic acid.

III. Conclusion

Cdk4 is a key factor that allows cells to pass through the G₁ phase checkpoint normally. The results of the present invention demonstrate that retinoic acid induced NTDs because it caused neural tube neuroepithelial cells to be arrested at the G₀/G₁ phase, which in turn resulted in a decrease in cell proliferation and caused unclosed or abnormally closed neural tubes, i.e., anencephaly or spina bifida. In normal embryos, Cdk4 expression in neural tube neuroepithelial cells was strong, indicating active cell proliferation. After the administration of retinoic acid, Cdk4 expression in neural tube neuroepithelial cells decreased, indicating that the majority of neural tube cells were unable to pass through the G₁ phase checkpoint, thus leading to NTDs. Pre-administration or early administration of GLSs appear to increase the expression of Cdk4 in neural tube neuroepithelial cells treated with retinoic acid, thus accelerating the neural tube cell proliferation and differentiation, so as to reduce the incidence of NTDs.

The results of the present invention further indicate that the administration of retinoic acid to female mammals prior to or at the early stage of the pregnancy lead to reduced transcription of Cdk2 mRNA and Cdk4 mRNA in embryonic neural tube neuroepithelial cells. As a result of the low expression of Cdk2 and Cdk4, a large number of cells were arrested at the G₀/G₁ phase and could not pass through the G₁ phase checkpoint. Consequently, the number of S phase cells was also reduced and a large number of cells were unable to differentiate and proliferate. Since there was only a small amount of nestin-positive cells (neural stem cells) in the neuroepithelial cells, differentiation and proliferation from these neural stem cells could not meet the needs of normal embryonic neural development. The result was that in the retinoic acid-treated pregnant mammals, a large number of embryos demonstrated serious NTDs. The crown-to-rump distance in these embryos was also significantly lower than that of normal embryos.

The administration of GLSs significantly reduced retinoic acid-induced embryonic NTDs. After administration of Ganoderma spores, Cdk4 mRNA transcription in embryonic neural tube epithelial cells was increased, G₀/G₁ phase cell ratio was reduced, S phase cell ratio was increased, and neural stem cell ratio was also notably increased.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for preventing or reducing a neural tube defect (NTD) in a mammalian embryo and/or fetus comprising:
administering to a female mammal capable of becoming pregnant an effective amount of germination activated sporoderm-broken Ganoderma lucidum spores (GLSs).; wherein said GLSs are administered to said female mammal prior to or at the early stage of the pregnancy; and wherein said NTD is caused by cell cycle arrest of a neuroepithelial cell in said embryo.

2. The method according to claim 1, wherein said GLSs prevent said neural tube defect in said mammalian embryo and/or fetus.

3. The method according to claim 1, wherein said GLSs reduces said neural tube defect in said mammalian embryo and/or fetus.

4. The method according to claim 1, wherein said NTD is anencephaly or spina bifida.

5. The method according to claim 1, wherein said female mammal is a human.

6. The method according to claim 5, wherein said GLSs are orally administered to said female mammal no later than 4 weeks of pregnancy.

7. The method according to claim 1, wherein said female mammal is a rodent.

8. The method according to claim 7, wherein cell cycle arrest is caused by retinoic acid.

9. The method according to claim 8, wherein said retinoic acid is all trans retinoic acid.

10. The method according to claim 1, wherein said cell cycle arrest is at a G₀/G₁ phase of a cell cycle.

11. The method according to claim 1, wherein said cell cycle arrest is caused by inhibition of cyclin-dependent kinase (Cdk).

12. The method according to claim 11, wherein said Cdk is Cdk4.

13. The method according to claim 1, wherein said GLSs are prepared by soaking Ganoderma spores in a solution which is selected from the group consisting of water, saline, and a nutritional solution to cause the spores to germinate; placing said germination-treated Ganoderma spores in a culture box at a relative humidity of 65-98% and temperature of 18-48° C. to cause the germinated Ganoderma spores to activate; and breaking sporoderm of said germination activated Ganoderma spores to produce said GLSs.

14. The method according to claim 1, wherein said effective amount of said GLSs is between 0.01 and 20 g/kg body weight/day.

15. The method according to claim 14, wherein said effective amount of said GLSs is between 0.1 and 20 g/kg body weight/day.

16. The method according to claim 11, wherein said GLSs increase Cdk4 expression in said neuroepithelial cells of said embryo.

17. The method according to claim 1, wherein said GLSs promote proliferation and differentiation of said neuroepithelial cells in said embryo.
